(19) United States
(12) Patent Application Publication
(10) Pub. No.: US 2009/0088782 A1
(43) Pub. Date: Apr. 2, 2009

(54) Flexible Spinal Rod With Elastomeric Jacket

(76) Inventors: Missoum Moumene, Newton, MA (US); Jonathan Fanger, Raynham, MA (US); Charles M. Bartish, JR., Providence, MA (US); Payman Afshari, South Easton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/863,867

(22) Filed: Sep. 28, 2007

(51) Int. Cl. A61B 17/08; A61B 17/58

(52) U.S. Cl. 606/151; 606/103

(57) ABSTRACT

A flexible rod for use in spinal stabilization having a tube having a helical slit defining a gap and an elastomeric jacket provided in the throughbore of the tube, within the gap, and upon the outer surface of the tube.

### BACKGROUND OF THE INVENTION

The vertebrae in a patient's spinal column are linked to one another by the disc and the facet joints, which control movement of the vertebrae relative to one another. Each vertebra has a pair of articulating surfaces located on the left side, and a pair of articulating surfaces located on the right side, and each pair includes a superior articular surface, which faces upward, and an inferior articular surface, which faces downward. Together the superior and inferior articular surfaces of adjacent vertebra form a facet joint. Facet joints are synovial joints, which means that each joint is surrounded by a capsule of connective tissue and produces a fluid to nourish and lubricate the joint. The joint surfaces are coated with cartilage allowing the joints to move or articulate relative to one another.

Diseased, degenerated, impaired, or otherwise painful facet joints and/or discs can require surgery to restore function to the three joint complex. Damaged, diseased levels in the spine were traditionally fused to one another. While such a technique may relieve pain, it effectively prevents motion between at least two vertebrae. As a result, additional stress may be applied to the adjoining levels, thereby potentially leading to further damage.

More recently, techniques have been developed to restore normal function to the facet joints. One such technique involves covering the facet joint with a cap to preserve the bony and articular structure. Capping techniques, however, are limited in use as they will not remove the source of the pain in osteoarthritic joints. Caps are also disadvantageous as they must be available in a variety of sizes and shapes to accommodate the wide variability in the anatomical morphology of the facets. Caps also have a tendency to loosen over time, potentially resulting in additional damage to the joint and/or the bone support structure containing the cap.

Other techniques for restoring the normal function to the posterior element involve arch replacement, in which superior and inferior prosthetic arches are implanted to extend across the vertebra typically between the spinous process. The arches can articulate relative to one another to replace the articulating function of the facet joints. One drawback of current articulating facet replacement devices, however, is that they require the facet joints to be resected. Moreover, alignment of the articulating surfaces with one another can be challenging.

Accordingly, there remains a need for improved systems and methods that are adapted to mimic the natural function of the facet joints.

US Patent Publication No. 2006/0142758 (“Petit”) discloses a linking element that consists of a helical spring and a support member made out of a polymer material. The helical spring is embedded in the support material.

US Patent Publication No. 2004/0215191 (“Kitchen”) discloses a flexible tube comprising at least one lumen that extends the length of the tube. At least one rod of a preformed curvature is present within said one lumen of the tube. As additional rods are placed within the hollow flexible member, increased force is applied to the spine by the device, thereby moving the spine towards the desired curvature.

US Patent Publication No. 2004/0049189 (“Le Couedic”) discloses a device that has two rigid rod-forming parts made of a first material. A connecting body that is made entirely from a second material that is more elastically deformable than said first material interconnects the two rod-forming portions.

US Patent Publication No. 2005/0065514 (“Studer”) discloses a dampening element comprising two spring elements coaxial with or parallel to a longitudinal axis, and two axially end-side connectors. The end-side connectors can be linked to the spring elements such that at least one of the spring elements is connected to the connectors. The two spring elements have different spring rates and one sprint element is designed as a tension and compression spring and comprises a spring coil, and the damping element is pre-stressed.

EP Patent Publication No. 0 677 277 (“Moreau I”) discloses a helically split oblong rotating member attached to upper and lower parts. The hollow central part of said member is filled at rest with a viscoelastic cushioning product cast in inter-thread overflow.

FR Patent Publication No. 2 717 370 (“Moreau II”) discloses an intervertebral stabilizing prosthesis comprising a hollow body of revolution that is radially and/or helically slotted to make it axially flexible, whose internal spaces and slots are filled with a viscoelastic product constituting an elastic shock-absorbing tensioner that is micrometrically adjustable. Yoke systems allow the assembly to be embedded by nuts into anchors and screwed into the bone.

GB Patent Publication No. 2 382 307 (“Sengupta”) discloses an assembly for soft stabilization of the spine comprising a pair of pedicle screws and a helical spring member. The helical spring member may be made from titanium or stainless steel. A plastic sleeve may or may not cover the spring.

US Patent Publication No. 2005/0203517 (“Jahng”) discloses an elastomer cladding on a wire.

### SUMMARY OF THE INVENTION

The device of the present invention provides a flexible rod used to provide dynamic stabilization of the spine when used with bone anchors. In preferred embodiments, the rod comprises a spring tube having a helical slit and an elastomeric polymer jacket. The spring tube allows elongation and subsequent changes in interpedicular distance and vertebral body rotation. The elastomeric jacket limits elongation, thus reducing strain on the tube.

One particularly preferred embodiment provides an elastomeric jacket that is injection molded around and within the spring tube containing the helical slit. Providing this design as a two-piece construction: a tube and an injection-molded elastomer. The rod design may also include two tips or caps at either end of the tube for MIS procedures, thereby making it a four-piece construction.

In some embodiments, there is provided a method of providing rods that rely on the radial compression of a central core elastomeric member through the helical tube component. Providing these rods of varying stiffness within one set to address various patient needs.

In some embodiments, there is provided an elastomeric member as a core within a helical tube component. This embodiment relies upon the helical tube and elongation of the elastomer to provide an elongation limit.

### DESCRIPTION OF THE FIGURES

FIG. 1A is a perspective view of the first embodiment of the rod of the present invention.

FIG. 1B is a first cross section of the first embodiment of the rod of the present invention.

FIG. 1C is a second cross section of the first embodiment of the rod of the present invention.

FIG. 1D is a side view of first embodiment of the rod of the present invention.

FIG. 2 is a cross section of the second embodiment of the rod of the present invention.

FIG. 3 is a cross section of the third embodiment of the rod of the present invention.

FIG. 4 is a cross section of the fourth embodiment of the rod of the present invention.

FIG. 5 is a cross section of the fifth embodiment of the rod of the present invention.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

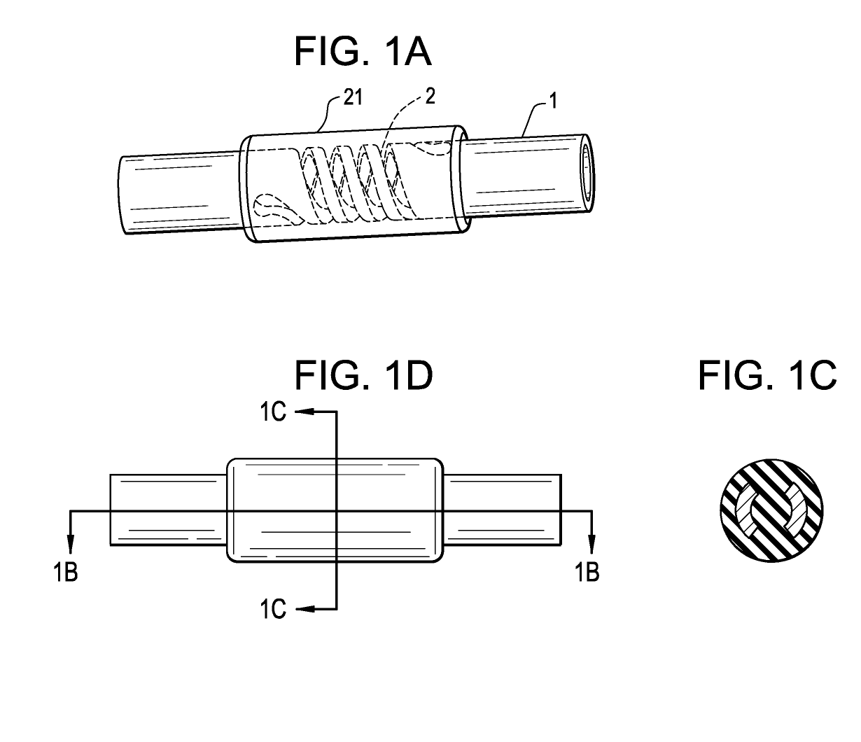

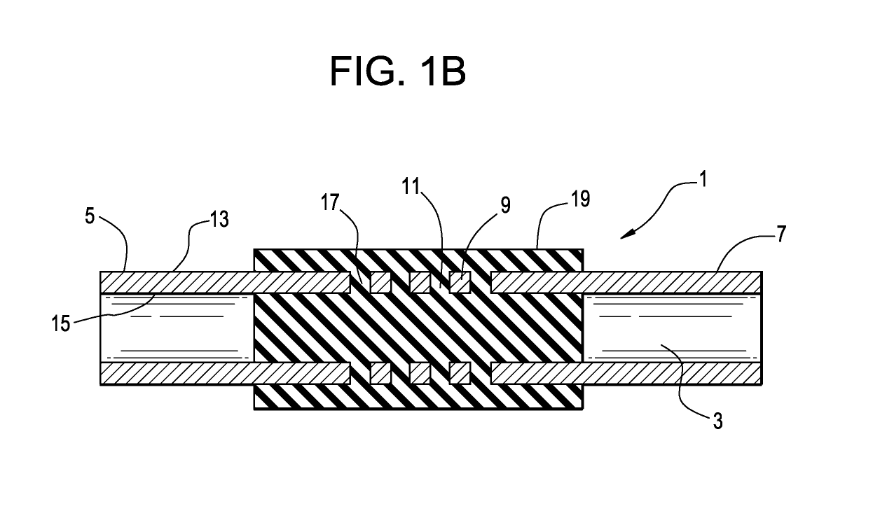

Now referring to FIG. 1, there is provided a flexible rod for use in spinal stabilization, the rod comprising:
a) a tube 1 having a throughbore 3, a first end portion 5, a second end portion 7, an intermediate portion 9 therebetween, wherein the intermediate portion comprises a helical slit 11 extending from an outer surface 13 of the tube to an inner surface 15 of the tube, the slit defining a gap 17 between the outer surface of the tube and the inner surface of the tube and a coil portion 2 of the tube, and
b) an elastomeric jacket 19 provided in the throughbore within the intermediate portion of the tube, within the gap, and upon the outer surface of the tube.
   The provision of the elastomeric jacket in each of the throughbore within the intermediate portion of the tube, within the gap, and upon the outer surface of the tube provides an advantageous locking of the jacket to the tube. Preferably, the jacket is injection molded so that it is continuous and integral between each of these regions.

In some embodiments (not shown), the rod further comprises c) a cap extending from the throughbore of each end portion of the tube. These caps (which preferably taper inwardly as they extend outwardly) allow for ease of rod insertion during MIS procedures. The caps may also contain features that allow attachment of an instrument used to guide the implant into position within the body.

In a second embodiments of the present invention, an elastomeric core is provided that optionally fills the gaps and at least part of the throughbore of the helical tube, but does not cover the outer surface of the tube. This type of core could extend the full length of the throughbore and be capped at the ends, such that the material would stretch with the helical cut rod. The cap could be a simple button or form a tip with a useful purpose, such as a contoured bullet tip for minimally invasive surgery. The advantage of this second embodiment is to utilize the caps as an extension stop/strain limiter. The caps could be flush with the end of the tube or a gap can be provided between the end of the tube and the cap to limit the elongation. The central core and caps together would connect both ends of the tube in an effort to prevent elongation, while creating a soft stop. The softness of stop could be influenced by factors such as clearance, elastic modulus, geometry of the core or durometer.

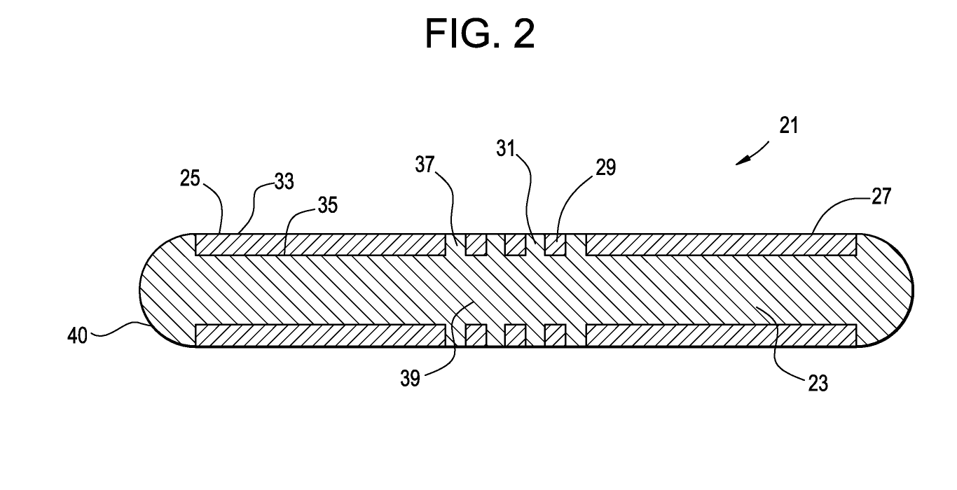

In regards to geometry, the core stiffness could be changed by changing its cross-section. In addition, an additional (preferably metallic) stiff core can be placed in the center of the elastomeric core to increase the rod stiffness in bending and shear loading. This rod can be connected to the two caps to act as an elongation limiter. Now referring to FIG. 2, there is provided the second embodiment of the present invention, which is a flexible rod for use in spinal stabilization, the rod comprising:
a) a tube 21 having a throughbore 23, a first end portion 25, a second end portion 27, an intermediate portion 29 therebetween, wherein the intermediate portion comprises a helical slit 31 extending from an outer surface 33 of the tube to an inner surface 35 of the tube, the slit defining a gap 37 between the outer surface of the tube and the inner surface of the tube, and
b) an elastomeric core 39 extending throughout the throughbore and gap of the tube and forming a cap 40 upon each end portion of the tube wherein the outer surface of the tube is free of elastomer.

In this embodiment, the cap shape could be a simple button or a tip with a useful purpose, such as a contoured bullet tip for minimally invasive surgery.

In a third embodiment of the present invention, an elastomeric core could be inserted into the tube and an elastomeric jacket could be tightly fitted around the outside of the core creating an additional elongation limit. This design would allow the gaps between the coils to be free from the elastomer. The advantage of this third embodiment is that the provision of the material-free gap prevents tears and inhibits tear propagation of the elastomeric core_during tube elongation.

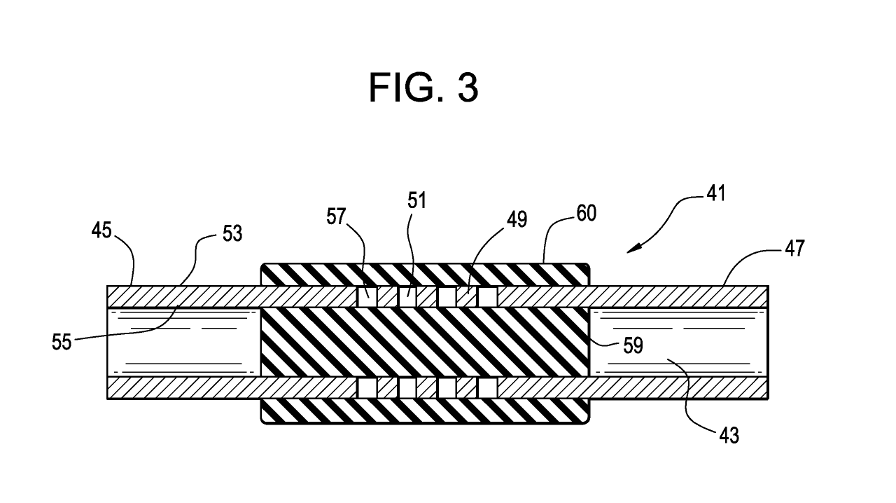

Now referring to FIG. 3, there is provided the third embodiment of the present invention, which is a flexible rod for use in spinal stabilization, the rod comprising:
a) a tube 41 having a throughbore 43, a first end portion 45, a second end portion 47, an intermediate portion 49 therebetween, wherein the intermediate portion comprises a helical slit 51 extending from an outer surface 53 of the tube to an inner surface 55 of the tube, the slit defining a gap 57 between the outer surface of the tube and the inner surface of the tube,
b) an elastomeric core 59 provided within the throughbore of the tube, and
c) an elastomeric jacket 60 provided upon the outer surface of the tube, the jacket covering at least the helical slit,
   wherein the gap of the tube is free of elastomer.

In a fourth embodiment of the present invention, an elastomeric core could be injected into the helical tube component taking care to keep the gaps in the coils free from the elastomer. Holes traversing the thickness of the tube are provided in its end portions. The holes allow elastomeric material to be injected into them. The advantages of this fourth embodiment are to lock the core to the tube in order to 1) provide a tube configuration that allows the elastomeric core to be formed to the tube without fear of migration; and 2) allow a specific amount of motion of the core relative to the tube before engaging the elastomer. The elastomeric material may fill the entire hole or it may only partially fill the hole such that the clearance allows coil motion of the tube. The holes traversing the thickness could take many shapes including notches, square cut outs, etc. The geometry of the core (straight diameter, tapered, necked down, etc) could be created to provide a tight fit with the inner diameter of the tube or allow specific elongation and stiffness requirements.

The core of this embodiment also provides additional stiffness for the assembly, hence relieving the tube with helical cuts from experiencing critical strains. The holes shape/location and numbers can be modified to obtain this desired effect.

Ends could also be molded into the elastomer, and these ends could be a simple button or form a tip with a useful purpose, such as a contoured bullet tip for minimally invasive surgery

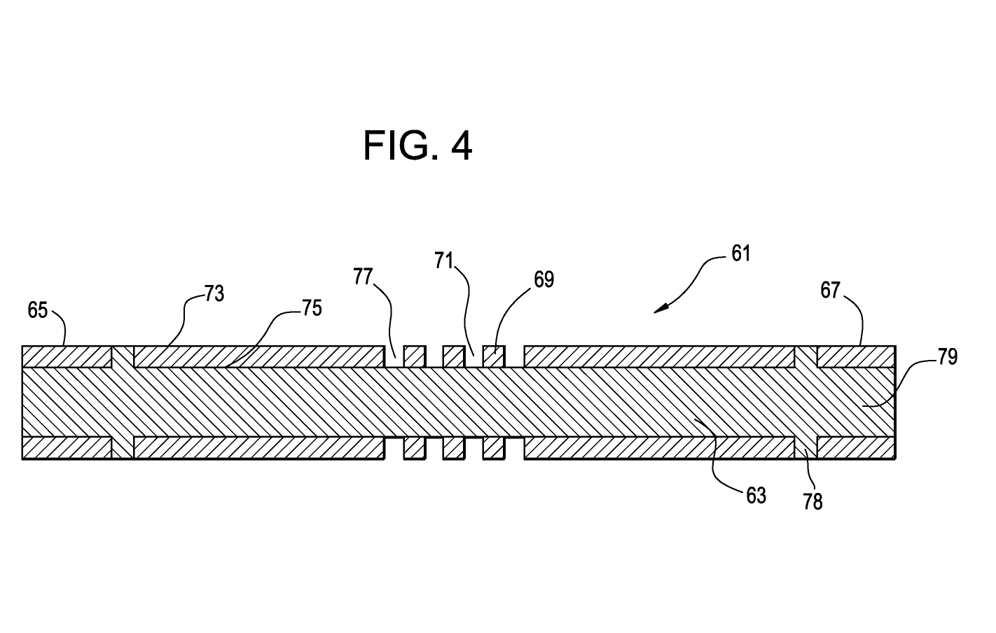

Now referring to FIG. 4, there is provided the fourth embodiment of the present invention, which is a flexible rod for use in spinal stabilization, the rod comprising:
a) a tube 61 having a throughbore 63, a first end portion 65, a second end portion 67, an intermediate portion 69 therebetween, wherein the intermediate portion comprises a helical slit 71 extending from an outer surface 73 of the tube to an inner surface 75 of the tube, the slit defining a gap 77 between the outer surface of the tube and the inner surface of the tube, each end portion having a plurality of holes 78 between the inner and outer surfaces,
b) an elastomeric core 79 provided within the throughbore of the tube and extending into the holes to lock the elastomer,
   wherein the gap and outer surface of the tube are free of elastomer.

As a fifth embodiment, instead of using voids to attach the elastomer to the tube (as in the fourth embodiment), the elastomeric core could have grooves at either end which mate with tabs extending inwardly from the inner surface of the tube and which fold into the groove. The advantage of this fifth embodiment is the same as the advantage of the fourth embodiment above, but with a different means to securely contain/attach the elastomeric core.

In addition, end features could also be molded into the elastomer, wherein the ends could be a simple button or form a tip with a useful purpose, such as a contoured bullet tip for minimally invasive surgery

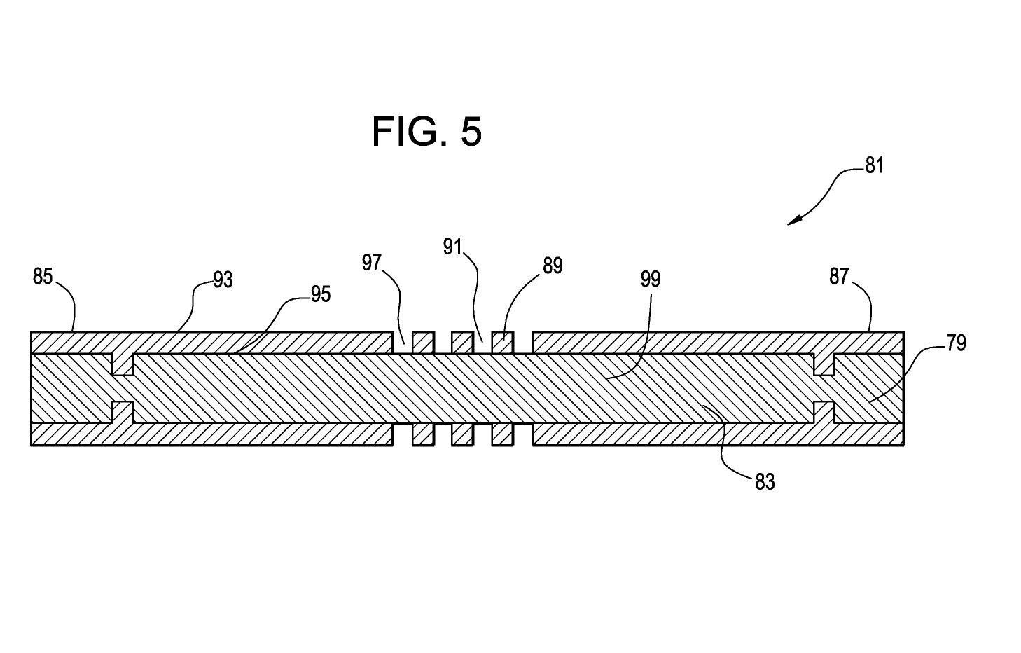

Now referring to FIG. 5, there is provided the fifth embodiment of the present invention, which is a flexible rod for use in spinal stabilization, the rod comprising:
a) a tube 81 having a throughbore 83, a first end portion 85, a second end portion 87, an intermediate portion 89 therebetween, wherein the intermediate portion comprises a helical slit 91 extending from an outer surface 93 of the tube to an inner surface 95 of the tube, the slit defining a gap 97 between the outer surface of the tube and the inner surface of the tube, each end portion having a plurality of tabs 98 extending inwardly from the inner surface,
b) an elastomeric core 99 provided within the throughbore of the tube and having grooves that interlock with the tabs of the tube,
   wherein the gap and outer surface of the tube are free of elastomer.

In a sixth embodiment of the present invention, an elastomeric core is fixed on at least one end of the tube. The invention is a kit of such rods providing cores with varying levels of clearance between the core and the coil, such that elongation of the helical tube is limited by the clearance between the core and coil. As the coil portion of the helical tube extends, it necks down at the center where it would contact the elastomeric core. This would reproduce motion that was closer to the neutral zone phenomenon, where motion is easier within the neutral zone, but becomes more restricted outside the neutral zone. A kit that provides various rods with a range of clearances between the core and helical coil to provide different levels of motion restriction within one implant set is desirable.

Now therefore, there is provided the sixth embodiment of the present invention, which is a kit of flexible rods for use in spinal stabilization, each rod comprising:
a) a tube having a throughbore having a diameter, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube and a coil, and
b) an elastomeric core having an outer surface and extending throughout the throughbore and being fixed to the first end portion of the tube,
wherein the outer surface of the core and the inner surface of the coil define a clearance,
   wherein a first rod and second rod have equal throughbore diameters and different clearances.

In a seventh embodiment of the present invention, there is a kit providing several rods having different axial stiffnesses within an implant system. Each different stiffness rod would be created with an elastomeric material that has a different elastic modulus. This would allow rods to be made for various patient needs in the event that certain patients require a greater range of motion or more limited motion. A material with a low modulus would be more likely to elongate/compress and a material with a high modulus would be less likely to elongate/compress.

Now therefore, there is provided the seventh embodiment of the present invention, which is a kit of flexible rods for use in spinal stabilization, each rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, and
b) an elastomeric core within the throughbore, the core having a diameter wherein a first core and a second core have equal diameters and different stiffnesses.

Elastomeric Jacket is injected around the tube and between the coils. The elastomeric jacket is shown semi-transparent to allow visualization of the tube and helix.

In some embodiments, the elastomeric jacket has a thickness of less than about 3 mm, preferably less than about 2 mm, more preferably about 1 mm.

Preferably, the elastomeric material completely fills the tube, the gaps within the coil and covers the outside of the tube.

In addition, the jacket will have a thickness that will require the helix to be placed between the screw heads, which will prevent the bone anchors from being locked on the helix region. This provides additional safety for the helical design.

The polymer jacket can preferably be formed from polycarbonate, but may also be formed of any other elastomeric biocompatible material depending on the properties desired. Generally, the polymer jacket is made of an elastomer, and may be preferably an elastomer as selected in U.S. Pat. No. 5,824,094 (“Serhan”). In some embodiments, the elastomeric jacket is preferably made of a polyolefin rubber or carbon black reinforced polyolefin rubber. The hardness of the elastomeric jacket may be preferably 56-72 shore A durometer. The ultimate tensile strength of the jacket may be preferably greater than 1600 psi. The jacket may have an ultimate elongation greater than 300% using the ASTM D412-87 testing method, and a tear resistance greater than 100 psi using the ASTM D624-86 testing method. Although the elastomeric jacket is disclosed as being made of a polyolefin rubber or polycarbonate in some embodiments, it can be made of any elastomeric material that simulates the characteristics of natural ligaments.

The helical tube is typically between about 20 mm_and 100 mm_in length. It generally has an outside diameter of between about_{—}3.5_and_{—}10_mm, and a thickness of between about_{—}0.4_ and_{—}3 mm.

The helical slit and coil are present within the intermediate portion of the tube, generally within the middle third to the middle and the middle fifth of the tube. The slit generally has a width of between_{—}0.25_and_{—}4_mm. The coil generally has a width of between _{—}0.5_and _{—}4_mm. Typically the slit has between about _{—}2_and _{—}10_ revolutions of the tube.

If a metal is chosen as the material of construction for the tube, then the metal is preferably selected from the group consisting of nitinol, titanium, titanium alloys (such as Ti-6Al-4V), chrome alloys (such as CrCo or Cr—Co—Mo) and stainless steel.

If a polymer is chosen as a material of construction for the tube, then the polymer is preferably selected from the group consisting of polycarbonates, polyesters, (particularly aromatic esters such as polyalkylene terephthalates, polyamides; polyalkenes; poly(vinyl fluoride); PTFE; polyarylethyl ketone PAEK; and mixtures thereof.

In some embodiments, the tube is made of a stainless steel alloy, preferably BioDur^{R} CCM Plus^{R} Alloy available from Carpenter Specialty Alloys, Carpenter Technology Corporation of Wyomissing, Pa. In some embodiments, the tube is made from a composite comprising carbon fiber. Composites comprising carbon fiber are advantageous in that they typically have a strength and stiffness that is superior to neat polymer materials such as a polyarylethyl ketone PAEK. In some embodiments, the tube is made from a polymer composite such as a PEKK-carbon fiber composite.

Preferably, the composite comprising carbon fiber further comprises a polymer. Preferably, the polymer is a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the carbon fiber comprises between 1 vol % and 60 vol % (more preferably, between 10 vol % and 50 vol %) of the composite. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite comprises:

a) 40-99% (more preferably, 60-80 vol %) polyarylethyl ketone (PAEK), and
b) 1-60% (more preferably, 20-40 vol %) carbon fiber,
wherein the polyarylethyl ketone (PAEK) is selected from the group consisting of polyetherether ketone (PEEK), polyether ketone ketone (PEKK) and polyether ketone (PEK).

In some embodiments, the composite consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

One skilled in the art will appreciate, however, that the rods may be configured for use with any type of bone anchor, e.g., bone screw or hook; mono-axial or polyaxial. Typically, a bone anchor assembly includes a bone screw, such as a pedicle screw, having a proximal head and a distal bone engaging portion, which may be an externally threaded screw shank. The bone screw assembly may also a receiving member that is configured to receive and couple a spinal fixation element, such as a spinal rod or spinal plate, to the bone anchor assembly.

The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, incorporated herein by reference. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, both of which are incorporated herein by reference.

Generally, in using the present invention, two bone anchors such as polyaxial screws are inserted into adjacent pedicles within a functional spinal unit of a patient. The flexible rod of the present invention is then inserted into the patient between the anchors. The first end portion of the flexible rod is attached to the first bone anchor and the second end portion of the flexible rod is attached to the second bone anchor. More preferably, this is achieved in a minimally invasive surgery.

We claim:

1. A flexible rod for use in spinal stabilization, the rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, and
b) an elastomeric jacket provided in the throughbore within the intermediate portion of the tube, within the gap, and upon the outer surface of the tube.

2. The rod of claim 1 wherein the elastomeric jacket comprises polycarbonate, and the tube comprises nitinol.

3. The rod of claim 1 wherein the elastomeric jacket forms a cap upon each end portion of the tube.

4. The rod of claim 1 further comprising:
c) a cap extending from the throughbore of at least one end portion of the tube.

5. A flexible rod for use in spinal stabilization, the rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, and
b) an elastomeric core extending throughout the throughbore and gap of the tube and forming a cap upon at least one end portion of the tube. (caps could be present without having elastomeric core filling gaps)

6. The rod of claim 5 wherein the outer surface of the tube is free of elastomer.

7. The rod of claim 5 wherein the elastomeric core comprises polycarbonate.

8. The rod of claim 5 wherein the tube comprises nitinol.

9. A flexible rod for use in spinal stabilization, the rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, and
b) an elastomeric core provided within the throughbore of the tube,
c) an elastomeric jacket provided upon the outer surface of the tube, the jacket covering at least the helical slit.

10. The rod of claim 9 wherein the gap of the tube is free of elastomer.

11. The rod of claim 9 wherein the elastomeric core comprises polycarbonate.

12. The rod of claim 9 wherein the tube comprises nitinol.

13. A flexible rod for use in spinal stabilization, the rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, each end portion having a plurality of holes between the inner and outer surfaces,
b) an elastomeric core provided within the throughbore of the tube and extending into the holes to securely attach the elastomer.

14. The rod of claim 13 wherein the gap of the tube is free of elastomer.

15. The rod of claim 13 wherein the outer surface of the tube is free of elastomer.

16. The rod of claim 13 wherein the elastomeric core comprises polycarbonate, and the tube comprises nitinol.

17. A flexible rod for use in spinal stabilization, the rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, each end portion having a plurality of tabs extending inwardly from the inner surface,
b) an elastomeric core provided within the throughbore of the tube and having grooves that interlock with the tabs/pins of the tube.

18. The rod of claim 17 wherein the gap and outer surface of the tube are free of elastomer.

19. A kit of flexible rods for use in spinal stabilization, each rod comprising:
a) a tube having a throughbore having a diameter, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube and a coil, and
b) an elastomeric core having an outer surface and extending throughout the throughbore and being fixed to the first end portion of the tube, wherein the outer surface of the core and the inner surface of the coil define a clearance,
wherein a first rod and second rod have equal throughbore diameters and different clearances.

20. A kit of flexible rods for use in spinal stabilization, each rod comprising:
a) a tube having a throughbore, a first end portion, a second end portion, an intermediate portion therebetween, wherein the intermediate portion comprises a helical slit extending from an outer surface of the tube to an inner surface of the tube, the slit defining a gap between the outer surface of the tube and the inner surface of the tube, and
b) an elastomeric core within the throughbore, the core having a diameter
wherein a first core and a second core have equal diameters and different stiffnesses.
